(19) United States
(12) Patent Application Publication
(10) Pub. No.: US 2008/0041397 A1
(43) Pub. Date: Feb. 21, 2008

(54) ARTICLE & METHOD FOR INDUCING PROPER BREATHING DURING SLEEP CYCLES TO REACTIVATE BODILY FUNCTIONS

(76) Inventor: Gene Hirs, Waterford, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/465,342

(22) Filed: Aug. 17, 2006

(51) Int. Cl. A61F 5/56

(52) U.S. Cl. 128/848

(57) ABSTRACT

A sleep assist article and associated method for establishing proper breathing patterns. An adhesive tape body is releasably secured upon a face of a person and exhibits an inner aperture defined by an inner edge surface, and through which upper and lower lips of the person extend. The body establishes a check valve in a first inhalation stage, to facilitate breathing through the person's nasal passages while inhibiting inhaling orally. The body further operates to permit exhaling orally, and while concurrently inhibiting nasal exhalation. The device optimizes an oxygen intake percentage within a user's lungs, as well as an associated oxygen saturation during repetitive breathing cycles associated with an induced sleep pattern and to improve sleep cycles, reduce incidences of sleep apnea, and to provide other health benefits.

### BACKGROUND OF THE INVENTION

1. Field of the Invention

The present inventions relate generally to articles and methods for inducing and maintaining proper sleep patterns in humans. In particular, the present invention teaches an article and associated method for optimizing a person's sleep cycle (in particular an older individual's), particularly as it relates to the individual's ability to absorb increased amounts of oxygen by virtue of the associated device's construction to encourage breathing in nasally (through the nose) and exhaling orally (through the mouth).

2. Description of the Prior Art

Incidences of shallow and improper breathing are becoming more commonplace as individuals age. Such irregular breathing cycles often are linked to physical and/or medical impediments as obesity, high blood pressure, arthritis, chronic fatigue, depression, infections, and sleep disturbances, such as sleep apnea.

This problem in large part is directly related to an individual's ability to circulate lymphatic fluids, as well as to deal with toxins and other waste material contained within such fluids. It is further medically known that a massaging effect can be created on the thoracic duct through deep breathing, and which moves the diaphragm downward, similar to a bellows, and in order to create a negative pressure within the lungs and chest cavity.

The efficiency of lymphatic circulation is, therefore, greatly improved by proper (e.g. deep) breathing and which functions to remove dead cells, viruses, bacteria, inorganic compounds, water, cholesterol, fats and other waste products. The purified lymph is, at that point, returned as blood plasma.

A further benefit of establishing proper breathing is in overcoming sleep apnea. This medically documented disturbance is created by the individual's tongue moving in a direction towards the back of the throat, thus resulting in a fatty tissue deposit at that region being pushed over the windpipe. As a result, the individual is caused to choke, typically resulting in tie person gasping and/or waking. In rare instances, coma or death can result from a sever apneatic episode.

It has further been documented that such apnea related incidents can occur upwards of sixty times per hour. Accordingly, maintaining an individual's own respiratory system under an appropriate and continuous air pressure and breathing is desired in order to eliminate this malady.

An example of a breathing assist apparatus (normalizer) is set forth in U.S. Patent Application Publication No. 2004/0211430, issued to Pivovarov, and which operates to prevent snoring, teeth grinding, and light forms of sleep apnea. The device includes an outer plate which is positioned external to tie user's mouth when in use, an elongated hollow shaft for connecting the structure to a lip plate adapted to be received between the user's lip and teeth, and a generally C-shaped multi-lobed structure adapted for receiving the user's tongue.

In use, the device is positioned within the oral cavity of the user in an operative configuration that the tongue is retained within the multi-lobed structure, the teeth clamping down upon the connector with the lip plate positioned between the teeth and the inner portions of the upper and lower lips. The outer plate further defines a centrally disposed chamber having an inlet tube in fluid communication with the hollow tubular connector for providing an inlet for breathing air.

The inlet tube is further adapted to connection to a source of gas, such as oxygen, to assist in delivering the gas to the user through the lungs. In addition, tie chamber includes a threaded peripheral edge adapted for threaded engagement with a container of medicine thereby facilitating the delivery of oral medications into the user's oral cavity and preferably the delivery of oral medications below the tongue. A medicine receiving chamber is farther provided to allow for medicine received therein to be dispensed and/or evaporated in the user's mouth. As a result of proper application of the apparatus breathing at night is normalized, while snoring, grinding of the teeth, and apnea are prevented, and further while medications may be simultaneously delivered orally.

Japanese Publication No. 2004/344431 discloses a breath assist device having a main body which is worn covering the mouth and a pair of ear wearing members mounted on both sides of the assist device main body, and which includes an inner projection projecting inward of the assist device main body. When the breath assist device is worn, the tip of the inner projection extends through between upper teeth and lower teeth toward the throat, allowing the mouth to be kept opened to prevent apnea.

U.S. Patent Application Publication No. 2005/0263152, to Fong, teaches isometric muscle manipulation resulting in a reduction in breathing airway restriction and the incidence and magnitude of sleep apnea and snoring. The method comprises exercises including raising the tip of the tongue upward and backward in the mouth to touch the area between the hard palate and the soft palate of the mouth and then pressing the tongue upwardly, raising the flat tongue upon it against the hard palate, placing the jaw in a slightly open attitude and pressing the tongue against the floor of the mouth, expanding the nasal and oral pharynx, and placing the jaw in the closed attitude and then protruding the jaw to a forward position.

### SUMMARY OF THE PRESENT INVENTION

A sleep assist article for establishing proper breathing patterns and which includes a body releasably secured upon a mouth of a person and including an inner aperture defined by an inner edge surface and through which upper and lower lips of the person extend. The body establishes a check valve effect in a first inhalation stage to facilitate inhaling through the person's nasal passages, and while inhibiting inhaling orally. The body further operates to permit exhaling orally, and while inhibiting nasal exhalation. The device solves the problem of arousals and sleep disturbances, including optimizing oxygen saturation in the body, reducing the systolic blood pressure due to less stressful conditions, lower cholesterol, loss of body fat and improving other body functions.

### BRIEF DESCRIPTION OF THE DRAWINGS

Reference will now be made to the attached drawings, when read in combination with the following description, wherein like reference numerals refer to like parts throughout the several views, and in which:

FIG. 1 is a side profile view illustrating the device according to the present inventions in an installed configuration and by which the user's lips are compressed, resulting in a nasal directed intake of air;

FIG. 2 is a successive side profile view illustrating the device according to the present inventions permitting the user's lips to partially open during an exhaling step;

FIG. 3 is a front plan view of the breath assist device according to the inventions and which includes a flexible and impermeable one-sided adhesive body, combined with a centrally disposed (lip seating and air passage) aperture, between which is optionally disposed an intermediate cloth layer;

FIG. 4 is a first diagrammatic illustration of percentage of total air intake in a person's lungs relative to inhale/exhale breathing cycle;

FIG. 5 is a second diagrammatic illustration of a ratio of oxygen saturation (by percent) relative to hours of sleep, both with and without tie breathing assist device according to the present inventions;

FIG. 6 is a third diagrammatic illustration of a frequency of sleep arousal indicating a number of sleep interruptions both with and without the device (e.g. 36:1) according to the present invention;

FIG. 7 is a bar graph illustration of pulse pressure factoring in systolic minus diastolic pressure readings for clinical studies including both assist and non-assist applications;

FIG. 8 is a further bar graph illustrating a cholesterol level after four months use of the breathing assist device according to the present invention; and

FIG. 9 is a weight loss graph illustrating weight reduction resulting from four months usage of the breathing assist device according to the present invention.

### DETAILED DESCRIPTION OF THE PREFERRED EMBODIMENTS

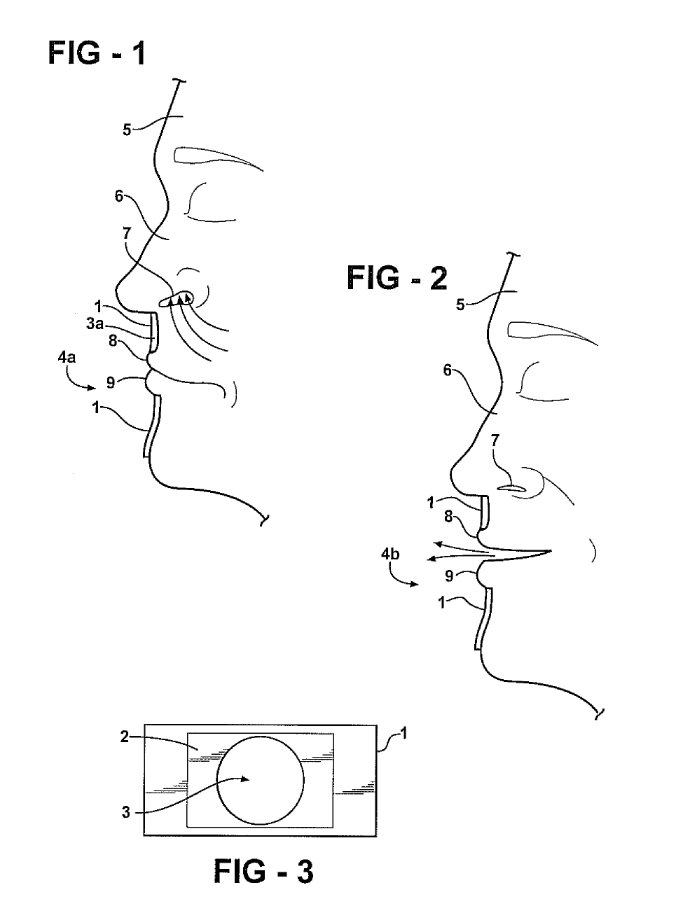

Referring now to FIGS. 1-3, in combination, an article and associated method is now described for assisting in maintaining proper breathing cycles of a person according to the present inventions. As previously described, the article and method of the present invention operates to optimize a person's sleep cycle (in particular older individual's), particularly as it relates to the individual's ability to absorb increased amounts of oxygen by virtue of the associated device's construction to encourage breathing in nasally (through the nose) and exhaling orally (through the mouth).

The breathing assist device, as best shown in FIG. 3, includes a flexible and impermeable one-sided adhesive body 1. An optional intermediate cloth or like fabric layer 2 may be overlaid, such as upon a central location of the body 1, and which may further exhibit a desired consistency and permeability. A centrally disposed (lip seating and air passage) aperture 3 is sized in an appropriate fashion and defines in inner continuous annular edge within the body 1. The aperture 3 may be formed within the optional intermediate cloth layer 2 or, as is also contemplated within the scope of the invention, may be formed directly in the body 1. The dimensions of the tape are further not limited to any specific size, but may exhibit any suitable shape and size including a central aperture 3 exhibiting an interior diameter of ⅞″.

FIG. 1 further illustrates, at 4a, a side profile outline of the device 1 according to the present inventions in an installed configuration upon a user's face 5 and which includes, in relevant part, a nose 6 with internal nasal passages (of which a single one 7 is illustrated in side profile) and a mouth defined by lips 8 and 9. In FIG. 1, die user's lips are compressed, owing to the substantially closed position associated with the compressing edge 3a of the inner aperture, thus resulting in a nasal directed inhalation of air. Reviewing further to FIG. 2, a successive side profile view 4b illustrates the device according to the present inventions during a subsequent exhalation step, and by which the geometry of the device 1 permits the user's lips (see at 3b) to partially open during an exhaling step.

By inhaling through the nose only, and exhaling orally (i.e. from the mouth), a proper sleep breathing cycle is established and so that breathing is regulated in both an automatic and natural fashion. The proper sequence of breathing can be further controlled if the user's mouth is permitted to exhale, but not inhale. In this condition, the nose is forced to perform the inhaling step by itself.

A further advantage of establishing a proper breathing cycle as outlined above is to avoid interruptions or choking in the individual's glotis. Otherwise, and in the instance of deep exhaling occurring through the user's nasal passages, air discharge would be blocked and choking and gasping by the individual would occur.

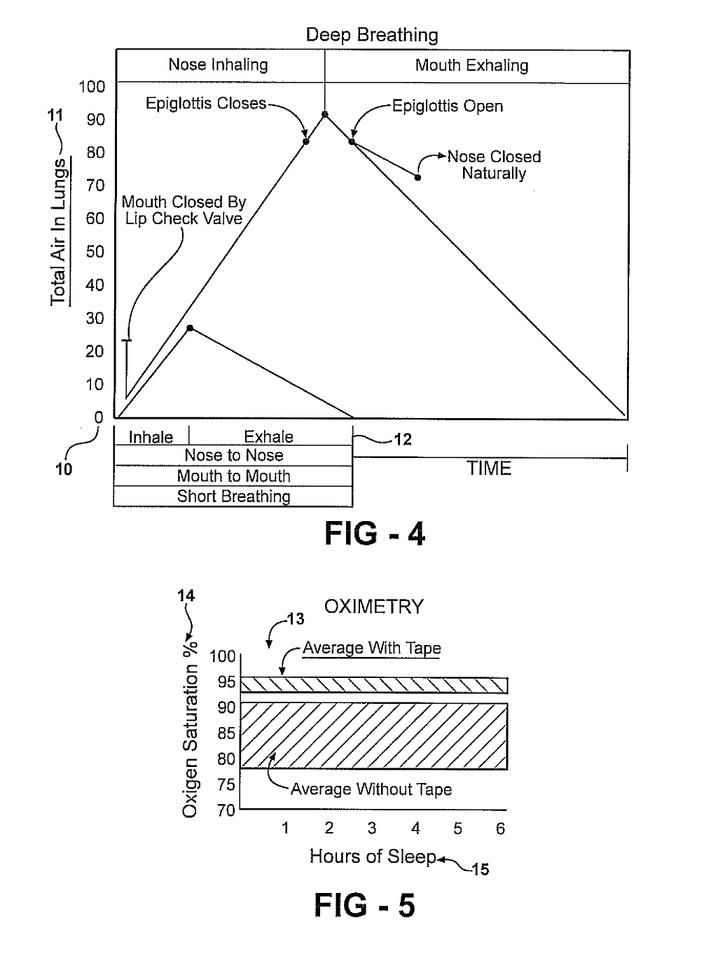

In support of the above, FIG. 4 presents a first diagrammatic illustration 10 of a percentage of total air intake 11 in a person's lungs relative to inhale/exhale breathing cycle 12. Specifically, the function of the lip closing check valve (see again aperture defining rim 3a in FIG. 1) during nasal inhalation greatly increases the total percentage of air intake 11 (up to 100% at the completion of the inhalation stage and immediately following closing of the individual's epiglottis, e.g. approximately at 90% air intake capacity). Following this, oral (mouth) exhalation is initiated and, following reopening of the epiglottis again at about 90%, the nasal passages close naturally and concluding at the end of the exhalation stage, at which point the lung air percentage is about 0% and immediately prior to the initiation of a subsequent cycle.

FIG. 5 presents at 13 a second diagrammatic illustration of a ratio of oxygen saturation (by percent) 14 relative to hours of sleep, as further represented at 15, both with and without the breathing assist device 1 according to the present inventions. In particular, FIG. 5 illustrates an average oxygen saturation of approximately 90% over a course of 1-6 hours of proper sleep, whereas a lowered average saturation range of 75-85 percent is typically encountered in test patients not utilizing the device 1.

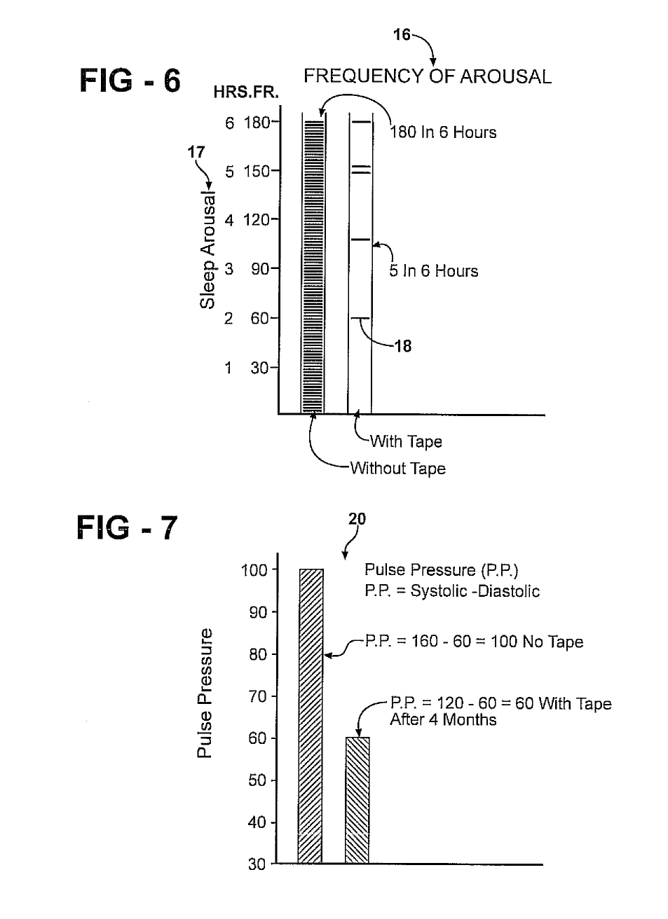

FIG. 6 further presents at 16 is a third diagrammatic illustration of a frequency of sleep arousal 17 relative to an indicated number of sleep interruptions 18, both with and without the device (e.g. 36:1) according to the present invention. This corresponds to a total of 180 interruptions occurring over a six hour period in individuals having sleep issues and not utilizing the sleep assist article 1, as opposed to a mere documented five (5) occurrences in individuals utilizing the sleep assist device 1.

FIG. 7 illustrates at 20 a bar graph illustration of pulse pressure factoring in systolic minus diastolic pressure readings for clinical studies including both assist and non-assist applications. In particular, clinical studies have established that the differences between systolic and diastolic pressures dropped forty (40) points for individuals after using the inhaling assist device for at least four months.

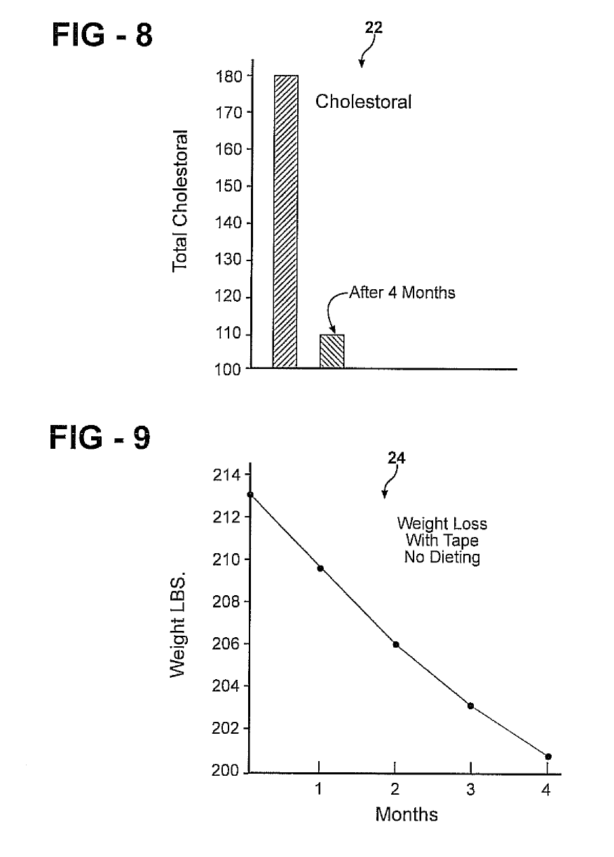

FIG. 8 illustrates at 22 a further bar graph illustrating a cholesterol level after four months use of the breathing assist device according to the present invention. Again, a drop (in this instance of 70 points) attended four months of continuous usage of the inhaling assist device.

Finally, FIG. 9 illustrates a weight loss graph 24 illustrating weight reduction, approx. 13 lbs, resulting from four months usage by an individual of the breathing/inhaling assist device according to the present invention.

An associated method is also disclosed for obtaining a proper and optimized breathing cycle according to the above article description. In particular, the method is accomplished through tie associated article operating as a check valve, such as when the user's lips are in a whistling (pursed) position by which air can escape from the mouth because it is pressurized. Inhalation is, in this stage, rendered impossible from the mouth, thus necessitating that the nasal passages initiate inhalation.

The present device and method provides benefits during both shallow breathing as well as proper/deep breathing cycles, as well as again assisting greatly in reducing the effects of sleep apnea. The benefits of more complete breathing include better lymphatic fluid circulation, as well as oxygen absorption which can be beneficial for weight loss, lowering of blood pressure, improving arthritic conditions, reducing chronic fatigue and other sleep disturbances, both apnea related and the like. Additional health issues the present device solves include arousals and sleep disturbances by optimizing oxygen saturation in the body, reducing the systolic blood pressure due to less stressful conditions, lower cholesterol, loss of body fat and improving other body functions.

In particular regards to sleep apnea, it has been found that this condition can be eliminated by changing ones breathing cycle, mainly by inhaling through the nose and exhaling from the mouth. As part of the desired methodology associated with the present invention, breathing must be rendered automatic and habit forming. This can be accomplished by practicing, such as twice daily for approximately fifteen minutes for three or four days and prior to installation of the breath assist tape at night before going to sleep. Upon installation, the user's lips must project through the central aperture formed in the tape and a breathing exercise initiated whereby the user keeps his/her mouth closed but allows air to escape during inhaling. Upon exhaling and closing the lips, inhalation is initiated through the nose and the cycle repeated.

Having described my invention, other and additional preferred embodiments will become apparent to those skilled in the art to which it pertains, and without deviating from the scope of the appended claims.

I claim:

1. A sleep assist article for establishing proper breathing patterns, comprising:
a body releasably secured upon a mouth of a person and including an inner aperture defined by an inner edge surface and through which upper and lower lips of the person extend; and
said body establishing a check valve effect in a first inhalation stage to facilitate inhaling through the person's nasal passages while inhibiting inhaling orally, said body further operating to permit exhaling orally, while inhibiting nasal exhalation;
said device solves the problem of arousals and sleep disturbances including optimizing oxygen saturation in the body, reducing the systolic blood pressure due to less stressful conditions, lower cholesterol, loss of body fat and improving other body functions.

2. The sleep assist article as described in claim 1, said body exhibiting a rectangular tape covering the mouth area.

3. The sleep assist article as described in claim 1, said aperture having a selected shape and size and further comprising a substantially circular inner edge.

4. The sleep assist article as described in claim 3, further comprising said circular inner edge defining an inner diameter of approximately ⅞″.

5. A method for establishing proper breathing patterns, comprising:
applying a body in releasably secured fashion upon a face of a person;
forming an inner aperture within said body defined by an inner edge surface and through which upper and lower lips of the person extend;
said body compressing against the user's lips to establish a check valve in a first inhalation stage, and to facilitate inhaling through the person's nasal passages while inhibiting inhaling orally; and
said body further operating to permit exhaling orally, while inhibiting nasal exhalation, during a subsequent exhalation stage;
said device optimizing an oxygen intake percentage within a user's lungs, as well as an associated oxygen saturation during repetitive breathing cycles associated with an induced sleep pattern.

6. The method as described in claim 5, said aperture having a selected shape and size and further comprising the step of defining a substantially circular inner edge.

7. The method as described in claim 6, further comprising the step of said circular inner edge defining an inner diameter of approximately ⅞″.

8. A sleep assist article, comprising:
a flexible body releasably secured over a mouth of a person and including an inner aperture through which extend the upper and lower lips of the person;
said body establishing a check valve effect in a first inhalation stage to facilitate breathing only through the person's nasal passages, said body further operating in a second oral exhalation stage while inhibiting nasal exhalation; and
said device optimizing at least one of an oxygen intake percentage within a user's lungs and an associated oxygen saturation during repetitive breathing cycles associated with an induced sleep pattern and in order to solve the problem of arousals and sleep disturbances including optimizing oxygen saturation in the body, reducing the systolic blood pressure due to less stressful conditions, lower cholesterol, loss of body fat and improving other body functions.
